(19) United States
(12) Patent Application Publication
(10) Pub. No.: US 2002/0016372 A1
(43) Pub. Date: Feb. 7, 2002

(54) Method for preventing and treating alzheimer's disease and brain damage associated with cardiov ascular disease and head injury

(76) Inventor: Anthony Clifford Allison, Belmont, CA (US)

Correspondence Address: Barbara J. Luther Chartered, 18124 Wedge Parkway, #516, Reno, NV, 89511, US

(21) Appl. No.: 09/879,498

(22) Filed: Jun. 11, 2001

Related U.S. Application Data
(60) Provisional application No. 60/222,143, filed on Jul. 31, 2000.

(51) Int. Cl. A61K 31/122

(52) U.S. Cl. 514/682

(57) ABSTRACT

A method for preventing and treating AD is supplementation with vitamin K so that the concentration of the vitamin in the circulation is sufficient for its functions outside the liver. Vitamin K supplementation will also be useful to reduce brain damage associated with cardiovascular disease and injury. Vitamin K can be administered orally, subcutaneously, intramuscularly or intravenously. The vitamin K can be phylloquinone, menaquinones of varying chain lengths, or menadione. Preferred forms of the vitamin are all-trans menadione and menaquinone-4. These are formulated so as to ensure efficient absorption from the gastrointestinal tract and rapid bioavailability in the brain following administration.

This application claims the benefit of the priority of U.S. Provisional Application No. 60/222,143, filed Jul. 31, 2000.

### BACKGROUND OF THE INVENTION

1. Field of Invention

The present invention relates to a method of medical treatment. More specifically the method provides for preventing the development of Alzheimer's disease (AD) in predisposed persons and for treatment of patients with dementias suggestive of AD. The treatment will also limit brain damage associated with cardiovascular disease and head injury.

2. Background Information and Related Disclosures

Alzheimer's disease (AD) is a type of dementia accompanied by characteristic pathological changes. The brains of patients with AD show extracellular deposits of β-amyloid protein, often associated with altered neurons or dendrites. Most cases also show within neurons neurofibrillary tangles composed of paired helical filaments containing a highly phosphorylated form of the tau protein of microtubules.

The prevalence of AD increases dramatically with age. In the U.S.A. 4% of persons 65 to 74 years of age were estimated to have AD; for those 75 to 84 years of age the prevalence rises to 16%; and among those 85 years or older to 48% (Evans, D. C. et al. Estimated Prevalence of Alzheimer's Disease in the United States. The Milbank Quarterly 68: 267-289, 1990). There are now about 4 million persons with AD in the U.S.A., and this figure is expected to rise substantially as the population ages. The projected number of cases in the year 2050 exceeds 14 millions.

In the great majority of AD patients there is no family history and the cause is unknown. Some early-onset patients have familial AD associated with known causes, including mutations in presenilin and β-amyloid precursor protein genes (Consensus Report of the Working Group on Molecular and Biochemical Markers of Alzheimer's Disease. Neurobiology of Aging 19: 109-116, 1998). Among the common genetic markers associated with AD, alleles of apolipoprotein E (APOE) are the most powerful and best documented. The e4 allele is a strong risk factor for the late-onset and sporadic forms of AD; whereas, the e2 allele protects against AD or at least delays its onset (Mayeux, R. et al. Utility of the Apolipoprotein E Genotype in the Diagnosis of Alzheimer's Disease. New England Journal of Medicine. 338: 506-511, 1998).

Primary drug therapies and emerging hormonal and investigational drug therapies have been reviewed by Flynn, B. I. (Pharmacologic Management of Alzheimer Disease. Annals of Pharmacotherapy 33: 178-187; 188-197; 840-849, 1999) and Knopman, D. S. (Advances in Methodology and Current Prospects for Primary Drug Therapies for Alzheimer's Disease. Methods in Molecular Medicine Vol. 32: Alzheimer's Disease: Methods and Protocols, edited by Hooper, N. M. Humana Press, Totowa, N.J., pages 45-60, 2000). The most widely used drugs are cholinesterase inhibitors, which have gastrointestinal side effects and narrow therapeutic windows. Although cholinesterase inhibitors significantly delay the decline in cognitive function in AD, these are palliative effects. Muscarinic agonists likewise delay the decline in cognitive performance in AD and reduce troublesome behaviors such as agitation, delusions and hallucinations: however, syncope and other peripheral side effects are common.

Some epidemiological studies have suggested that estrogen replacement, the antioxidant tocopherol and nonsteroidal anti-inflammatory drugs may prevent AD; and separate intervention trials with these agents are in progress. According to Flynn and Knopman (loc. cit.) the data are insufficient to recommend any of these agents for preventing AD or as a primary therapy of AD.

Because the survival of cholinergic neurons in the basal forebrain is dependent on nerve growth factor (NGF), attempts have been made to administer small molecules that stimulate NGF activity. Idebenone increases NGF levels in the brains of aged rodents, and appeared to have some activity in European trials on AD patients: however, a U.S. trial was abandoned because no activity was found in idobenone-treated patients. Knopman (loc. cit.) concludes: “One can only hope that medications that are more potent than the cholinesterase inhibitors can be developed.”

The search for new pathogenic mechanisms in AD and for new therapeutic strategies continues. The treatments listed above are the subject of numerous disclosures and papers. There have been no disclosures associating vitamin K with AD or the prevention of brain damage associated with cardiovascular disease.

### SUMMARY OF THE INVENTION

In one embodiment, there is disclosed a method of treating Alzheimer's Disease which calls for administering vitamin K in a dose which is sufficient to provide optimal extrahepatic activity of vitamin K. The vitamin K is administered orally, subcutaneously, intramuscularly or intravenously. The vitamin K may be phylloquinone, menaquinones of varying chain lengths, or menadione. Preferably, the menaquinone is menaquinone-4. Preferably the menadione is all-trans menadione.

In another embodiment, there is disclosed a method of preventing Alzheimer's Disease, preferably in individuals with the apolipoprotein e4 allele. This method includes administering an amount of vitamin K sufficient to maintain extrahepatic activity. Preferably, the vitamin K is administered orally and is menaquinone-4 or all-trans menadione.

In another embodiment, there is disclosed a method of treating cerebrovascular accidents which calls for administering vitamin K in a dose which is sufficient for optimal extrahepatic activity. Preferably, the vitamin K is menaquinone-4 or all-trans menadione and is administered intravenously.

In yet another embodiment, there is disclosed a method of preventing brain atrophy caused by cerebrovascular disease, which entails administering vitamin K in a dose, which is sufficient for extrahepatic activity. The vitamin K is administered orally, subcutaneously, intramuscularly or intravenously. Preferably the vitamin K is menaquinone-4 or all-trans menadione.

### DETAILED DESCRIPTION OF THE INVENTION

The present invention describes a method to prevent the onset of AD in predisposed persons and to treat patients with dementias suggestive of AD. According to the present invention a relative deficiency of vitamin K in ageing humans contributes to the pathogenesis of AD. Dietary supplementation with vitamin K will prevent AD and be useful for treating persons with this progressive disease. Dietary vitamin K supplementation will also reduce brain damage associated with cardiovascular disease.

The general designation vitamin K is given to several related compounds sharing a naphthoquinone ring structure with a methyl group at position 2 and an aliphatic side chain at position 3. Members of the vitamin K group differ in the length and degree of saturation of the side chain. Phylloquinone (vitamin K₁) has an aliphatic side chain of four prenyl residues, the first of which is unsaturated. In menaquinone (vitamin K₂) the number of prenyl residues may vary from 4 to 13 and all are unsaturated. The 2-methylnaphthoquinone ring without a side chain, termed menadione, has also been used therapeutically. This ring cannot be synthesized by animals and is a dietary requirement. All forms of vitamin K can restore hemostasis in vitamin K-deficient animals.

The biochemical mechanism by which vitamin K promotes hemostasis is well established. In the liver prothrombin and some other proteins are post-translationally modified by a vitamin K-dependent enzyme reaction: glutamate (Glu) is converted to γ-carboxyglutamate (Gla). The vicinal carboxyl groups in Gla bind calcium with high affinity, and calcium is required for the activity of the enzyme cascade leading to blood coagulation. Coumarin-type oral anticoagulants, such as warfarin, block the regeneration of vitamin K from the corresponding epoxide, thereby inhibiting the γ-carboxylation of proteins required for blood coagulation.

More recently, it has been established that vitamin K-dependent γ-carboxylation of Glu occurs also in extrahepatic sites and modifies proteins with other functions. One such protein is osteocalcin, which is produced by osteoblasts and is a marker of bone formation. Gamma carboxylation of Glu increases the binding of osteocalcin to hydroxyapatite. Vitamin K deficiency occurs frequently in postmenopausal women and elderly men, even though their blood coagulation is normal. This suggests that the requirement of vitamin K for extrahepatic actions is higher than that which is adequate for its hepatic actions. The relative vitamin K deficiency in older humans is associated with undercarboxylated osteocalcin, osteoporosis and increased risk of hip fractures (Vermeer, C. et al. Role of vitamin K in Metabolic Bone Disease. Annual Review of Nutrition 15: 1-22, 1995; Tamatani, M. et al. Decreased Levels of Vitamin K and 25-hydroxyvitamin D in Osteopenic Elderly Men. Metabolism 47: 195-199, 1998). The association of vitamin K deficiency with increased risk of hip fractures has been observed consistently in several studies in three continents, and thus is firmly established.

Another protein in which γ-carboxylation of Glu occurs is matrix Gla protein: this protein appears to be involved in removal of calcium from arterial walls and from growing cartilage. Mice with targeted disruption of the matrix Gla protein gene have massive deposition of calcium in their aortas and die a few weeks after birth of hemorrhage from aortic aneurysms. Neonatal rats treated with warfarin likewise show early and massive deposition of calcium in their aortas and coronary valves. In humans aortic calcification is associated with decreased vitamin K intake and undercarboxylated osteocalcin. These findings have led to the proposal that vitamin K supplementation in older humans may decrease the risk of osteoporosis as well as calcification of the aorta and coronary arteries, with associated risk of cardiovascular disease. Not wishing to be bound by any theory, it is now proposed that a relative deficiency of vitamin K in ageing humans could also affect brain function.

For the purposes of this invention, vitamin K deficiency is redefined on the basis of insufficient extrahepatic activity, not on the basis of impaired blood coagulation. Using that criterion, a relative deficiency of vitamin K is frequent in elderly human populations, both females and males. The same relative deficiency could also affect other extrahepatic activities of vitamin K, including those in the brain.

Vitamin K in plasma is bound to chylomicrons and chylomicron remnants carrying apolipoprotein E (APOE). Clearance of chylomicrons and their remnants from the circulation depends on binding of APOE to a hepatic receptor. Clearance is fast in people with one or two copies of genes encoding variant e4 and slow in those with e2. In consequence, plasma phylloquinone levels are strongly influenced by APOE genotype, being highest in e2, intermediate in e3and lowest in e4 (Saupe, J. et al. Phylloquinone Transport and its Influence on γ-carboxyglutamate Residues of Osteocalcin in Patients on Hemodialysis. American Journal of Clinical Nutrition 58: 204-209, 1993; Kohlmeier, M. et al. Transport of Vitamin K to Bones in Humans. Journal of Nutrition 126 supplement: S1192-1196, 1996). In the latter two genotypes increased levels of undercarboxylated osteocalcin have been observed, indicating extrahepatic vitamin K deficiency. The functional counterpart of this relative vitamin K deficiency is the finding, in women aged 65 years or older, that those with the e4 allele have a significantly increased risk of osteoporotic hip fractures as compared with those bearing other APOE alleles (Cauley, J. A. et al. Apolipoprotein E Polymorphism: a New Genetic Marker of Hip Fracture Risk—the Study of Osteoporotic Fractures. Journal of Bone and Mineral Research 14: 1175-1181, 1999; Johnston, J. M. et al. APOE 4 and Hip Fracture Risk in a Community-based Study of Older Adults. Journal of the American Geriatric Society 47: 1342-1345, 1999). Thus, the APOE genotype with lowest levels of circulating phylloquinone (e4) is most susceptible to AD; whereas, the genotype with the highest levels of phylloquinone (e2) is least susceptible.

The distribution of different forms of vitamin K in several organs of the rat has been analyzed (Thijssen, H. H. W. and Drittij-Reijnders, M. J. Vitamin K Distribution in Rat Tissues: Dietary Phylloquinone is a Source of Tissue Menaquinone-4. British Journal of Nutrition 72: 415-425, 1994). The predominant form in the liver is phylloquinone. However, in the brain and some other organs, levels of phylloquinone were found to be low while that of menaquinone-4 was higher. Dietary supplementation with phylloquinone increased the concentration of menaquinone-4 in the brain. Mammalian tissues are known to have the capacity to prenylate menadione to menaquinone-4 (Dialmieh, G. H. et al. Isolation and Characterization of menaquinone-4 as a Product of Menadione Metabolism in Chicks and Rats. International Journal of Vitamin and Nutrition Research 41: 391-400, 1971). At least in the rat, the predominant form of vitamin K in the brain is menaquinone-4. The level is moderately high (much higher than in blood), and can be augmented by dietary vitamin K supplementation.

The use of coumarin derivatives in pregnancy is associated with considerable risk for fetal sequelae (Pauli, R. M. and Haun, J. M. Intrauterine Effects of Coumarin Derivatives. Developmental Brain Dysfunction 66: 229-247, 1993). Exposure during the first trimester may result in warfarin embryopathy, manifested by nasal hypoplasia and epiphyseal calcification. These are attributed to the known effects of vitamin K on bone formation and calcium metabolism. In addition, exposure of women to coumarin derivatives, particularly in the second trimester, can result in abnormalities in the fetal central nervous system and mental retardation. The pathogenesis of these abnormalities is unknown, but it is unlikely that they are always secondary to hemorrhage. The association of maternal exposure to coumarin derivatives with abnormalities in the fetal central nervous system further supports my thesis that vitamin K is required for normal brain development and function.

Treatment of mice with warfarin was found to decrease brain sulfotransferase activity in vivo, as well as the incorporation of labeled sulfate into brain sulfatides (Sundaram, K. S. and Lev, M. Regulation of Sulfotransferase Activity by Vitamin K in Mouse Brain. Archives of Biochemistry and Biophysics 277: 109-113, 1990). Conversely, supplementation of untreated mice with phylloquinone or menadione increased brain sulfotransferase activity. Heretofore, this finding has not been applied to brain function or to the pathogenesis of AD.

Keratan sulfate (KS) is a linear polymer of β1,3-linked N-acetyl-lactosamine (Galβ1, 4GlcNAc) disaccharide units that are sulfated to a variable degree on the C-6 positions of either the glucosamine or galactose residues. Chemical analysis showed that KS, in contrast to heparan sulfate, is dramatically decreased in the cerebral cortex of AD patients (Lindahl, B. et al. Selective Loss of Cerebral Keratan Sulfate in Alzheimer's Disease. Journal of Biological Chemistry 271: 16991-16994, 1996). This finding was paralleled by the loss in AD of epitopes in the cell bodies of cortical neurons recognized by a monoclonal antibody with specificity for highly sulfated KS. Loss of cerebral KS was not seen in age-matched controls without AD, arguing against this being a general effect of ageing. The authors conclude that the specific staining of highly sulfated KS structures in normal neurons, and their lack in AD neurons, may reflect a specific functional defect in this particular disease. Defective sulfation of KS in AD could result from subnormal activity of a specific brain sulfotransferase, conceivably secondary to a deficiency of vitamin K in the brain.

Sulfated proteoglycans are found within several cell types and on their surfaces, where they can be components of adhesion molecules. Keratan sulfate proteoglycans show remarkable changes in the developing brain. A major protein of synaptic vesicles (SV-2) is a keratan sulfate proteoglycan (Scranton, T. W. et al. The SV-2 Protein of Synaptic Vesicles is a Keratan Sulfate Proteoglycan. Journal of Neurochemistry 61: 29-44, 1993). Cloned SV-2 shows sequence similarities to neurotransmitter transporters, and SV-2 has been implicated in acetylcholine transport. Immunocytochemical studies localize SV-2 proteoglycan and synaptophysin to synapses in normal human brain. There is a loss of both proteins in cortical AD neurons, consistent with a loss of synaptic function. In AD SV-2 proteoglycan was largely confined to neuritic plaques in dystrophic neurons. Thus, one manifestation of decreased sulfation in AD could be abnormal structure and function of the SV-2 protein in synaptic vesicles.

Another vitamin K-dependent protein in the brain is Gas 6, a product of growth arrest specific gene 6. Both Gas 6 and its tyrosine kinase receptor are widely distributed throughout the central nervous system (Prieto, A. L. et al. Gas 6, a Ligand for the Receptor Tyrosine Kinase Tyro 3, is Widely Expressed in the Central Nervous System. Brain Research 816: 646-661, 1999). Gas 6 is post-translationally modified by vitamin K-dependent γ-carboxylation of Glu, and Gla residues are required for its activity (Nakano, T. et al. Requirement of γ-Carboxyglutamic Acid Residues for the Biological Activity of Gas 6: Contribution of Endogenous Gas 6 to the Proliferation of Vascular Smooth Muscle Cells. Biochemical Journal 323: 387-392, 1977). Interaction of Gas 6 with its tyrosine kinase receptor (Axl) is a growth stimulus for several cell types, including smooth muscle cells (Nakano et al., loc. cit.) and Schwann cells. Gas 6 also prevents apoptosis of neurons. The mouse homologue of the receptor tyrosine kinase Axl is termed Ark. Gas 6/ Ark signaling occurs in neurons through the serine-threonine kinase Akt, a downstream component of the phosphoinositide-3-kinase (P13-K) pathway (Allen, M. P. et al. Growth Arrest-specific Gene 6 (gas 6)/Adhesion-related Kinase (Ark) Signaling promotes Gonadotropin-releasing Hormone Neuronal Survival via Extracellular Signal-related Kinase (ERK) and Akt. Molecular Endocrinology 13: 191-201, 1999). Activation of Akt promotes neuronal survival by phosphorylating and inactivating components of the apoptotic pathway; in addition, activation of Akt results in phosphorylation of a forkhead transcription factor and its retention in the cytoplasm (Brunet, A., et al. Akt Promotes Cell Survival by Phosphorylating and Inhibiting a Forkhead Transcription Factor. Cell 96: 857-868, 1999). The unphosphorylated forkhead transcription factor enters the nucleus and transcribes Fas ligand, which mediates apoptosis.

Addition of vitamin K to the chick embryo increases protein tyrosine phosphorylation in the brain, whereas warfarin decreases it (Saxena, S. P. et al. A Novel Role for Vitamin K₁ in a Tyrosine Phosphorylation Cascade during Chick Embryogenesis. Journal of Clinical Investigation 99: 602-607, 1977). This phosphorylation cascade occurs through c-Eyk, a chicken counterpart of the receptor tyrosine kinase family; substrates include focal adhesion kinase (pp 125^{FAK}), the cytoskeletal protein paxillin and pp 60^{src}. The authors propose that vitamin K plays an important role in the development of the central nervous system. In a review (Tsaioun, K. I. Vitamin K-dependent Proteins in the Developing and Aging Nervous System. Nutrition Reviews 57: 231-240, 1999), it is likewise suggested that vitamin K-dependent processes contribute to age-related changes in the central nervous system. Tsaioun concludes: “the study of the effects of the dietary vitamin K on the CNS functioning in populations of different age, gender and disease states will facilitate the development of a concept for optimal nutrient intake for specific population groups with relation to vitamin K nutrition.” While these general statements are valid, they do not focus specifically on AD, or correlate inherited predisposition to AD with vitamin K transport, as herein proposed. The role of neuronal apoptosis in the pathogenesis of AD has been widely discussed, and the observation that the vitamin K-dependent Gas 6-tyrosine kinase receptor interaction can protect neurons against apoptosis (Allen et al.; Brunet et al., loc. cit.) is highly relevant.

The neurofibrillary tangles in AD contain a highly phosphorylated form of the microtubule-binding protein Tau. Menadione induces dephosphorylation of Tau in neuroblastoma cells (Ko, L. et al. Menadione-induced Tau Dephosphorylation in Cultured Human Neuroblastoma Cells. Brain Research 760: 118-128, 1997). It is reasonable to postulate that vitamin K deficiency contributes to hyper-phosphorylation of Tau in AD.

All these observations lead to my thesis that a relative deficiency of vitamin K contributes to the pathogenesis of AD. Therefore, a method of preventing AD in predisposed persons and treating established cases of AD includes supplementation with vitamin K.

Turning to the disorder of vascular disease, quantitative magnetic resonance imaging (MRI) findings on 396 surviving members of the National Heart, Lung and Blood Institute Twin Study were analyzed according to the extent of vascular disease and Apo E allele status (De Carli, C. et al. Impact of Apolipoprotein E4 and Vascular Disease on Brain Morphology in Men from the NHLBI Twin Study. Stroke 30: 1548-1553, 1999). Carriers of e4 had significantly smaller brain volumes than those with other APOE genotypes. The co-occurrence of cerebrovascular disease and e4 was associated with significantly more brain atrophy and white-matter hyper-intensity than were observed with either e4 or cerebrovascular disease alone. The authors conclude that Apo E4 enhances the extent of brain abnormalities in the presence of various vascular diseases. This effect may be mediated by an increased susceptibility to brain injury or impaired repair mechanisms associated with Apo E4. The proposal is now made that a blood-borne factor, the concentration of which is low in persons with Apo E4, decreases susceptibility to and/or improves repair after brain injury. A further proposal is that the missing factor is vitamin K, in which case supplementation with the vitamin may decrease brain damage produced by cerebrovascular disease. Prophylactic administration of the vitamin in older, susceptible persons, and therapeutic administration following cerebral thrombosis or hemorrhage, would therefore be justified.

The most convenient form of prophylaxis with vitamin K is an orally administered formulation that is well absorbed. In some persons, when absorption from the intestine is inefficient, parenteral administration of the vitamin may be necessary. Parenteral administration will also rapidly increase levels of vitamin K in the brain when needed, for example following cerebral thrombosis or hemorrhage. Since vitamin K may limit the spread of brain damage beyond sites initially affected, the vitamin may also be useful in head injuries.

Any formulation of vitamin K that increases the concentration of the vitamin in extrahepatic sites will be suitable for the prevention and treatment of AD. Tablets, such as Mephyton® (Merck & Co., West Point, Pa.), have been used successfully to restore blood coagulation. Mixed micellar formulations of vitamin K, such as Konakion® MM (Roche, Nutley, N.J.), which are well absorbed from the gastrointestinal tract, are also suitable. Aqueous colloidal forms of vitamin K, such as AquaMephyton® (Merck & Co.), are injectable and may be useful when absorption from the intestine is inefficient.

Specifically included in the definition of vitamin K are all the different forms of vitamin K, including but not limited to K₁, K₂, and K₃. Examples of forms of vitamin K that may be useful to prevent and treat AD are phylloquinone, menaquinones of varying chain lengths, and menadione. Because menaquinone-4 is present in the brain, this form of the vitamin may be particularly suitable for preventing and treating AD and other brain disorders. However, it not known whether menaquinone-4 in the circulation enters the central nervous system unchanged. Dietary phylloquinone augments brain levels of menaquinone-4 (Thijssen, H. H. W. and Drittij-Reinders, M. J. Vitamin K Distribution in Rat Tissues: Dietary Phylloquinone is a Source of Tissue Menaquinone-4. British Journal of Nutrition 72: 415-425, 1994). In the rat menaquinone-4 is derived from menadione (Dalmieh, G. H. et al. Isolation and Characterization of Menaquinone-4 as a Product of Menadione Metabolism in Chickens and Rats. International Journal of Vitamin and Nutrition Research 41: 391-400, 1971). Presumably the saturated aliphatic side chain in phylloquinones is removed and replaced by a four-residue unsaturated chain to generate the form of vitamin K used in the brain and some other tissues.

In this invention, menadione itself is a preferred precursor because it is easily synthesized and metabolically modified. However, stereospecificity is an important consideration. The naturally occurring 2-methylnaphthoquinone is all-trans and this is the form which is metabolized to menaquinone-4. Because of the potential side effects of menadione it is highly desirable to administer therapeutically only the all-trans isomer. Like other quinones, menadione has oxidant effects, and can deplete thiol-containing compounds in cells and perturb calcium-mediated signaling (Karczowski, J. M. Prevention of Oxidant-induced Cell Death in Caco-2 Colon Carcinoma Cells after Inhibition of Poly (ADP-ribose) polymerase and Ca²⁺ chelation: involvement of a common mechanism. Biochemical Pharmacology 57: 19-26, 1999). Menadione can induce DNA damage (Woods, D. A. Measurement of Menadione-induced DNA Damage in Human Lymphocytes Using the Comet Assay. Free Radical Research 26: 113-124, 1997). Moreover, there has been concern that vitamin K administration may be associated with childhood cancer (discussed by Passmore, S. J. et al. Case-control Studies of Relation Between Childhood Cancer and Neonatal Vitamin K Administration. British Medical Journal 316: 184-189, 1988). Using only the all-trans isomer of menadione minimizes such risks.

Also included in this invention are vitamin K salts, such as sodium bisulfite, sodium diphosphate, and dimethylpyrimidinol bisulfite. There is no limitation on how the vitamin K form may be obtained; for example, vitamin K may be isolated from plants or bacteria, chemically synthesized (e.g., menadione and phytonadione), or prepared by a combination of both methods. However, if the compound is synthesized, it is desirable to separate the isomers by methods well known in the art and administer only the all-trans isomer.

Vitamin K may be provided in oral or parenteral forms. The oral form is usually more convenient. However, in forgetful patients in whom compliance is questionable, the parenteral form is preferred. The parenteral form is also preferred in persons with malabsorption problems or on antibiotic treatment. Among the different parenteral delivery modes, intramuscular or subcutaneous administration is preferred, as it establishes a long-term depot for the gradual release of vitamin K; however, any method of administration can be used. For example, in individuals who are fed intravenously or enterally, a salt of vitamin K is conveniently added to the feeding solution.

The adult daily requirement to maintain blood coagulation is typically about 2 mg. For treatment of hypoprothrombinemia in patients taking anticoagulants, 5-10 mg of oral phytonadione is indicated. The usual adult IM dose is 10 mg. To prevent osteoporosis, a daily oral dose of 40 mg menaquinone-4 has been administered. To prevent Alzheimer's disease, a daily oral dose of 40 mg menaquinone-4 is preferred. However, daily doses of any form of vitamin K in the range 1 to 100 mg may prove to be suitable.

Other brain disorders that can be treated by the inventive method include, but are not limited to, cerebrovascular accidents, cerebrovascular ischemia and head injury. Intravenous vitamin K injection is particularly preferred after cerebrovascular accidents and head injury, as it increases the level of vitamin K most rapidly. Following one or more intravenous doses, oral maintenance doses of vitamin K are indicated. Both intravenous and maintenance doses in the range of 1 to 100 mg per day are suitable.

1. A method of treating Alzheimer's Disease comprising administering vitamin K in a dose which is sufficient to provide optimal extrahepatic activity.

2. The method of claim 1 wherein the vitamin K is administered orally, subcutaneously, intramuscularly, sublingually or intravenously.

3. The method of claim 1 wherein the vitamin K is phylloquinone, menaquinones of varying chain lengths, or menadione.

4. The method of claim 3 wherein the menadione is all-trans menadione.

5. The method of claim 3 wherein the menaquinone is menaquinone-4.

6. A method of preventing Alzheimer's Disease comprising administering an amount of vitamin K sufficient to provide extrahepatic activity.

7. The method of claim 5 wherein vitamin K is administered orally and is all-trans menaquinone.

8. The method of claim 6 wherein vitamin K is administered orally and is menaquinone-4.

9. A method of treating cerebrovascular accidents comprising administering vitamin K in a dose which is sufficient to provide optimal extrahepatic activity.

10. The method of claim 9 wherein the vitamin K is an all-trans menadione and is administered intravenously.

11. The method of claim 9 wherein the vitamin K is menaquinone-4.

12. A method of preventing brain atrophy caused by cerebrovascular disease comprising administering vitamin K in a dose which is sufficient to provide optimal extrahepatic activity.

13. The method of claim 12 wherein the vitamin K is administered orally, subcutaneously, intramuscularly or intravenously.

14. The method of claim 12 wherein the vitamin K is phylloquinone, menaquinones of varying chain lengths, or menadione.

15. The method of claim 14 wherein the menadione is all-trans menadione.

16. The method of claim 14 wherein the menaquinone is menaquinone-4.

17. A method for preventing the spread of brain damage following head injury by the administration of all-trans menadione or menaquinone-4.
